(19) United States
(12) Patent Application Publication
(10) Pub. No.: US 2002/0142963 A1
(43) Pub. Date: Oct. 3, 2002

(54) Methods for increasing bone formation and enhancing bone accretion

(76) Inventor: Dennis L. Andress, Seattle, WA (US)

Correspondence Address: CHIRON CORPORATION, Intellectual Property - R440, P.O. Box 8097, Emeryville, CA, 94662-8097, US

(21) Appl. No.: 10/001,913

(22) Filed: Oct. 24, 2001

Related U.S. Application Data
(60) Provisional application No. 60/243,054, filed on Oct. 24, 2000.

(51) Int. Cl. A61K 38/30

(52) U.S. Cl. 514/12

(57) ABSTRACT

Methods of enhancing bone formation and/or bone accretion in vivo are disclosed. The methods utilize IGFBP-5 polypeptides, including full-length IGFBP-5, analogs or fragments thereof. These molecules can be delivered alone or in combination with agents which inhibit bone resorption or additional agents that enhance bone accumulation.

### CROSS-REFERENCE TO RELATED APPLICATION

This application is related to provisional patent application Ser. No. 60/243,054 filed Oct. 24, 2000, from which application priority is claimed under 35 USC §119(e)(1) and which application is incorporated herein by reference in its entirety.

This invention was made with support under NIH Grant R01-AR4491 from the National Institutes of Health, U.S. Department of Health and Human Services. Accordingly, the United States Government may have certain rights in the invention.

### TECHNICAL FIELD

The instant invention relates generally to polypeptide factors and their use in treating and preventing bone disorders. Specifically, the invention relates to the use of insulin-like binding protein-5 (IGFBP-5) polypeptides to increase bone formation and/or enhance bone accretion in vivo.

### BACKGROUND OF THE INVENTION

Insulin-like growth factors (IGFs) are low molecular weight polypeptide hormones with structural homology to proinsulin. Two different IGFs are known, IGF-I and IGF-II. These molecules are mitogenic in vitro for a wide variety of cells in tissue culture. Both IGFs stimulate in vitro growth of various tissues and induce collagen synthesis. IGF-I mediates the growth promoting effect of growth hormone in chondrogenesis and bone formation. Thus, IGF-I is essential for normal growth of an individual. IGF-II is believed to play a key role in fetal development and nerve growth.

IGFs stimulate cell growth in a variety of tissues including bone. The growth stimulatory effects of IGF-I and IGF-II in bone have been shown for cells of osteoblast lineage. Because both IGFs are secreted by this cell type and are recoverable from bone matrix, they are capable of affecting osteoblast function through autocrine and paracrine mechanisms. Factors that regulate IGF effects in osteoblasts are not completely understood.

The activity of the IGFs in bone and other tissues is mediated in part by a family of structurally related proteins that specifically bind to the IGFs, known as IGF binding proteins (“IGFBPs”). At least six major species of IGFBPs have been identified, designated IGFBP-1 through IGFBP-6, respectively, based on nomenclature agreed upon at the 2^{nd} International IGF symposium (1991). The six IGFBPs are reviewed and compared in Hwa et al., Endocrine Rev. (1999) 20:761-787.

The existence of a number of different IGFBPs indicates that these proteins have different functions. Indeed, despite similarities in amino acid sequence between the various IGFBPs, their effects on the growth of bone cells differ. In particular, IGFBPs either inhibit or potentiate IGF effects on osteoblasts. IGFBP-5, cloned first by Kiefer et al., Biochem. Biophys. Res. Commun. (1991) 176:219-225 (termed “IGFBP-6” in the publication), is the most abundant IGFBP found in bone. IGFBP-5 is present in bone matrix and is secreted by osteoblasts where it functions to stimulate osteoblast activity in vitro. See, e.g., Andress and Birnbaum, Biochem. Biophys. Res. Commun. (1991) 176:213-218. A recombinant, truncated IGFBP-5, including amino acids 1-169 of the mature molecule (IGFBP-5¹⁻¹⁶⁹), has been shown to stimulate the proliferation of osteoblasts in vitro by a mechanism that is independent of IGF-1 receptor binding. This effect may be mediated by a recently identified serine kinase receptor that specifically binds IGFBP-5. Andress, D. L., Am. J Physiol. (1998) 274:E744-E750. IGFBP-5 has also been shown to increase alkaline phosphatase activity and osteocalcin levels in a dose-dependent manner in vivo. Richman et al., Endocrinology (1999) 140:4699-4705.

Osteoporosis is caused by a reduction in bone mineral density in mature bone and results in fractures after minimal trauma. The disease is widespread and has a tremendous economic impact. The most common fractures occur in the vertebrae, distal radius (Colles' fracture) and hip. An estimated one-third of the female population over age 65 will have vertebral fractures, caused in part by osteoporosis. Moreover, hip fractures are likely to occur in about one in every three woman and one in every six men by extreme old age.

Two distinct phases of bone loss have been identified. One is a slow, age-related process that occurs in both genders and begins at about age 35. This phase has a similar rate in both genders and results in losses of similar amounts of cortical and cancellous bone. Cortical bone predominates in the appendicular skeleton while cancellous bone is concentrated in the axial skeleton, particularly the vertebrae, as well as in the ends of long bones. Osteoporosis caused by age-related bone loss is known as Type II osteoporosis.

The other type of bone loss is accelerated, seen in postmenopausal women and is caused by estrogen deficiency. This phase results in a disproportionate loss of cancellous bone, particularly trabecular bone. Osteoporosis due to estrogen depletion is known as Type I osteoporosis. The main clinical manifestations of Type I osteoporosis are vertebral, hip and Colles' fractures. The skeletal sites of these manifestations both contain large amounts of trabecular bone. Bone turnover is usually high in Type I osteoporosis. Bone resorption is increased but there is inadequate compensatory bone formation.

It is readily apparent, therefore, that methods for preserving bone mineral density, such as by increasing the rate of bone formation and enhancing accretion, especially in Type I osteoporosis, would be highly desirable.

### DISCLOSURE OF THE INVENTION

The present invention is based on the discovery that IGFBP-5 polypeptides, including full-length IGFBP-5, analogs and fragments thereof, are able to increase bone formation and/or enhance bone accretion in vivo. These molecules can be delivered alone or in combination with agents which inhibit bone resorption and/or enhance bone formation.

Accordingly, in one embodiment, the subject invention is directed to a method for increasing bone formation and/or enhancing bone accretion in a subject who has Type I osteoporosis. The method comprises administering to the subject a pharmaceutically effective amount of an IGFBP-5 polypeptide.

In certain embodiments, the IGFBP-5 polypeptide is a full-length IGFBP-5 molecule, such as the IGFBP-5 polypeptide depicted at positions 1-252, inclusive of FIGS. 1A-1B, or a molecule with 75% identity thereto. In other embodiments, the IGFBP-5 is a C-terminally truncated fragment of the IGFBP-5 depicted at amino acid positions 1-252, inclusive, of FIGS. 1A-1B, or a C-terminally truncated fragment of a molecule with 75% identity to the IGFBP-5 depicted at amino acid positions 1-252, inclusive, in FIGS. 1A-1B. The C-terminal truncation occurs at an amino acid after amino acid 100, or after amino acid 143 or 150. In another embodiment, the C-terminally truncated fragment of the IGFBP-5 comprises amino acids 1-169, inclusive, of FIGS. 1A-1B, or a sequence of amino acids with 75% identity to the contiguous sequence of amino acids 1-169 of FIGS. 1A-1B.

In another embodiment, the invention is directed to a method for increasing bone formation and/or enhancing bone accretion in a subject who has estrogen deficiency-related Type I osteoporosis, which comprises administering to the subject a pharmaceutically effective amount of a polynucleotide encoding an IGFBP-5 polypeptide.

These and other aspects of the present invention will become evident upon reference to the following detailed description and attached drawings. In addition, various references are set forth herein which describe in more detail certain procedures or compositions, and are therefore incorporated by reference in their entirety.

### BRIEF DESCRIPTION OF THE FIGURES

FIGS. 1A and 1B (SEQ ID NOS:1 and 2) depict a representative full-length cDNA and corresponding amino acid sequence for human IGFBP-5. The mature, full-length sequence begins at amino acid position 1 (nucleotide position 61) and ends at position 252 (nucleotide position 816). The negative numbers, −20 through −1, represent a signal sequence.

FIG. 2 shows bone mineral density (BMD) measurements of femur as a function of weeks of treatment in control ovariectomized mice (ovx-pbs), as well as ovariectomized mice administered full-length recombinant IGFBP-5 (intact) and C-terminally truncated IGFBP-5 (1-169).

FIG. 3 shows bone mineral density (BMD) measurements of spine as a function of weeks of treatment in control ovariectomized mice (ovx-pbs), as well as ovariectomized mice administered full-length recombinant IGFBP-5 (intact) and C-terminally truncated IGFBP-5 (1-169).

FIG. 4 shows bone mineral density (BMD) measurements of femur after four weeks of treatment in sham operated mice administered PBS (sham PBS), control ovariectomized mice (OVX PBS), as well as ovariectomized mice administered full-length recombinant IGFBP-5 (OVX intact) and C-terminally truncated IGFBP-5 (OVX 1-169).

FIG. 5 shows bone mineral density (BMD) measurements of spine after four weeks of treatment in sham operated mice administered PBS (sham PBS), control ovariectomized mice (OVX PBS), as well as ovariectomized mice administered full-length recombinant IGFBP-5 (OVX intact) and C-terminally truncated IGFBP-5 (OVX 1-169).

FIG. 6 shows bone mineral density (BMD) measurements of femur after eight weeks of treatment in sham operated mice administered PBS (sham PBS), control ovariectomized mice (OVX PBS), as well as ovariectomized mice administered full-length recombinant IGFBP-5 (OVX intact) and C-terminally truncated IGFBP-5 (OVX 1 -169).

FIG. 7 shows bone mineral density (BMD) measurements of spine after eight weeks of treatment in sham operated mice administered PBS (sham PBS), control ovariectomized mice (OVX PBS), as well as ovariectomized mice administered full-length recombinant IGFBP-5 (OVX intact) and C-terminally truncated IGFBP-5 (OVX 1-169).

FIG. 8 shows bone formation rate (BFR) measurements of femur after eight weeks of treatment in sham operated mice administered PBS (sham PBS), control ovariectomized mice (OVX PBS), as well as ovariectomized mice administered full-length recombinant IGFBP-5 (OVX intact) and C-terminally truncated IGFBP-5 (OVX 1 -169).

FIG. 9 shows bone formation rate (BFR) measurements of spine after eight weeks of treatment in sham operated mice administered PBS (sham PBS), control ovariectomized mice (OVX PBS), as well as ovariectomized mice administered full-length recombinant IGFBP-5 (OVX intact) and C-terminally truncated IGFBP-5 (OVX 1-169).

FIG. 10 shows osteoblast surface measurements of femur after eight weeks of treatment in sham operated mice administered PBS (sham PBS), control ovariectomized mice (OVX PBS), as well as ovariectomized mice administered full-length recombinant IGFBP-5 (OVX intact) and C-terminally truncated IGFBP-5 (OVX 1-169).

FIG. 11 shows osteoblast surface measurements of spine after eight weeks of treatment in sham operated mice administered PBS (sham PBS), control ovariectomized mice (OVX PBS), as well as ovariectomized mice administered full-length recombinant IGFBP-5 (OVX intact) and C-terminally truncated IGFBP-5 (OVX 1-169).

FIG. 12 shows osteoclast measurements of femur after eight weeks of treatment in sham operated mice administered PBS (sham PBS), control ovariectomized mice (OVX PBS), as well as ovariectomized mice administered full-length recombinant IGFBP-5 (OVX intact) and C-terminally truncated IGFBP-5 (OVX 1-169).

FIG. 13 shows osteoclast measurements of spine after eight weeks of treatment in sham operated mice administered PBS (sham PBS), control ovariectomized mice (OVX PBS), as well as ovariectomized mice administered full-length recombinant IGFBP-5 (OVX intact) and C-terminally truncated IGFBP-5 (OVX 1-169).

### DETAILED DESCRIPTION OF THE INVENTION

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990).

All publications, patents and patent applications cited herein, whether supra or infra, are hereby incorporated by reference in their entirety.

The following amino acid abbreviations are used throughout the text:

| | |
|---|---|
| Alanine: Ala (A) | Arginine: Arg (R) |
| Asparagine: Asn (N) | Aspartic acid: Asp (D) |
| Cysteine: Cys (C) | Glutamine: Gln (Q) |
| Glutamic acid: Glu (E) | Glycine: Gly (G) |
| Histidine: His (H) | Isoleucine: Ile (I) |
| Leucine: Leu (L) | Lysine: Lys (K) |
| Methionine: Met (M) | Phenylalanine: Phe (F) |
| Proline: Pro (P) | Serine: Ser (S) |
| Threonine: Thr (T) | Tryptophan: Trp (W) |
| Tyrosine: Tyr (Y) | Valine: Val (V) |

I. Definitions

In describing the present invention, the following terms will be employed, and are intended to be defined as indicated below.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification.

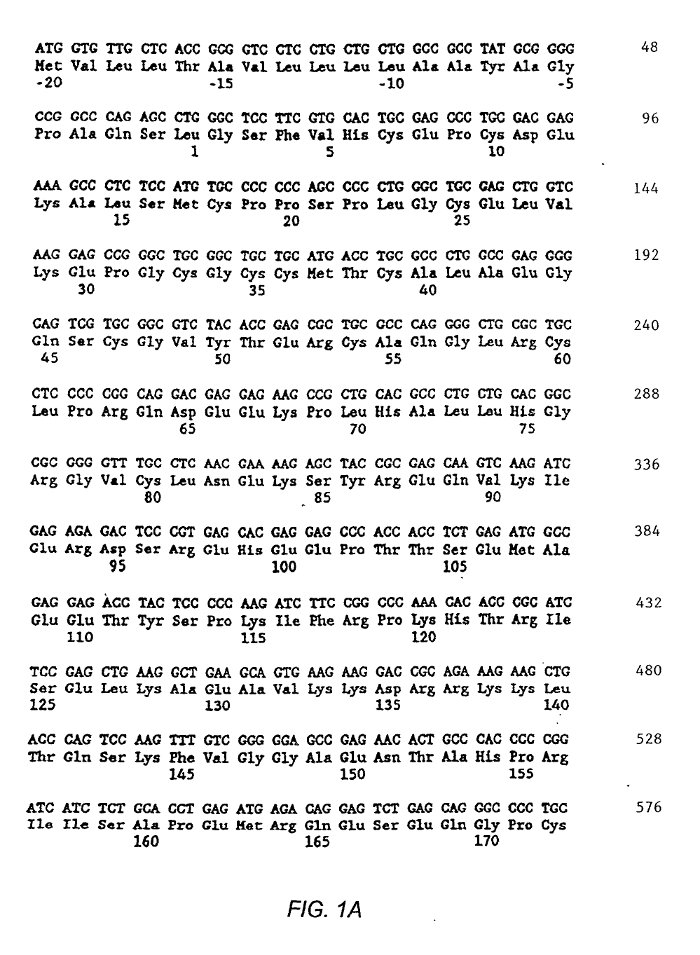

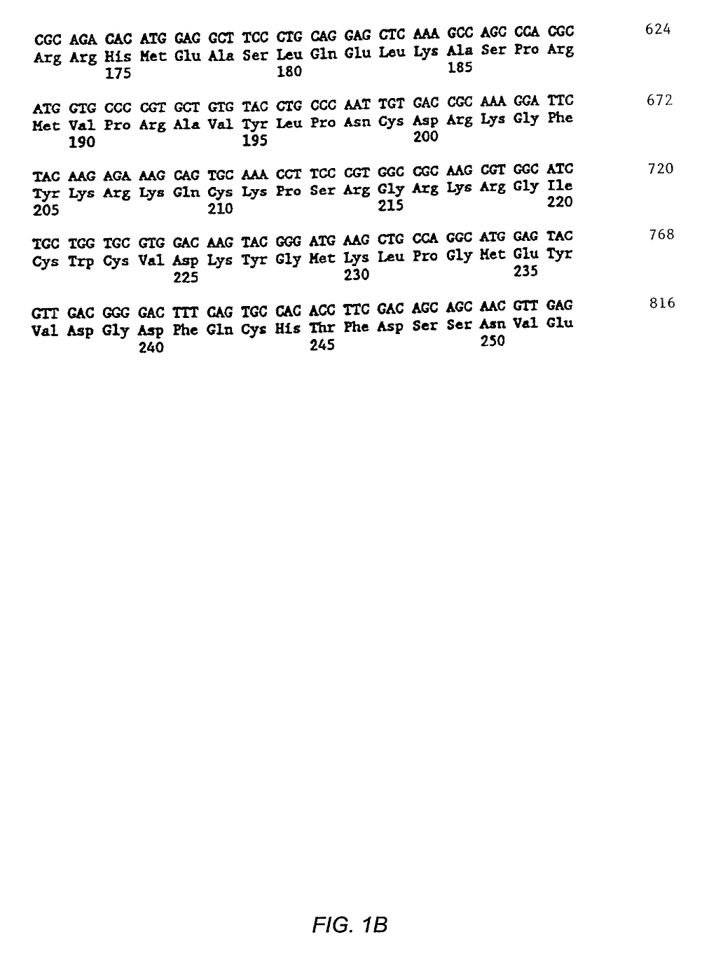

By “IGFBP-5 polypeptide” is meant the native, human, full-length IGFBP-5 shown at positions 1-252 of FIGS. 1A-1B, as well as biologically active fragments, variants, analogs and muteins thereof, that retain the ability to increase the bone formation rate and/or enhance bone accretion in vivo, as determined using any of several techniques well known in the art. For example, bone accretion can be determined using an animal model, such as an ovariectomized mouse, dog and the like. The animal is administered the test compound and bone mineral density (BMD) measured in bones that are normally depleted in Type I osteoporosis, such as bones of the axial skeleton, particularly the spine including the vertebrae, as well as in the ends of long bones, such as the femur, midradius and distal radius. Spinal measurements are particularly preferred as Type I osteoporosis primarily affects trabecular bone of the spine. Several methods for determining BMD are known in the art. For example, BMD measurements may be done using, e.g., dual energy xray absorptiometry or quantitative computed tomography, and the like. (See, the examples.) Similarly, increased bone formation can be determined using methods well known in the art. For example, dynamic measurements of bone formation rate (BFR) can be performed on tetracycline labeled cancellous bone from the lumbar spine and distal femur metaphysis using quantitative digitized morphometry (see, e.g., Ling et al., Endocrinology (1999) 140:5780-5788. Alternatively, bone formation markers, such as alkaline phosphatase activity (see, e.g., Farley et al., Calcif. Tissue Int. (1992) 50:67-73) and serum osteocalcin levels (see, e.g., Taylor et al., Metabolism (1988) 37:872-877 and Baylink et al., 10^{th} Annual Congress of Endocrinology, San Francisco, Calif. (1996) Abstract PI-945), can be assessed to indirectly determine whether increased bone formation has occurred.

The IGFBP-5 polypeptide may be synthetically or recombinantly produced. Moreover, the IGFBP-5 polypeptide may be isolated from natural sources, such as from any of several tissues of any mammalian source, for example human, rodent, bovine, canine, equine, ovine, porcine, etc. The sequences for various IGFBP-5 proteins are known. See, e.g., International Publication No. WO 92/14834, published Sep. 3, 1992, for the rat and human IGFBP-5 sequences, as well as FIGS. 1A-1B herein for the human IGFBP-5 sequence. The mammalian IGFBP-5 sequences appear to be highly conserved. For example, the rat and human amino acid sequences display approximately 97% homology to each other.

The terms “analog” and “mutein” refer to biologically active derivatives of the reference molecule, or fragments of such derivatives, that retain desired activity, as described above. In general, the term “analog” refers to compounds having a native polypeptide sequence and structure with one or more amino acid additions, substitutions (generally conservative in nature) and/or deletions, relative to the native molecule, so long as the modifications do not destroy immunogenic activity. The term “mutein” refers to peptides having one or more peptide mimics (“peptoids”), such as those described in International Publication No. WO 91/04282. Preferably, the analog or mutein has at least the same biological activity as the parent molecule, and may even display enhanced activity over the parent molecule. Methods for making polypeptide analogs and muteins are known in the art and are described further below.

Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 1-70 conservative or non-conservative amino acid substitutions, such as 5-50, 15-25, or any integer between 1-70, so long as the desired function of the molecule remains intact. One of skill in the art may readily determine regions of the molecule of interest that can be modified with a reasonable likelihood of retaining biological activity as defined herein. In general, amino acids occurring at positions 100 through the C-terminus, more preferably amino acids 143 through the C-terminus, will tolerate change without changing the function of the molecule. Preferably, changes to the amino acid sequence will preserve the native amino acids found at positions 130-143. One of skill in the art can readily determine other regions that will tolerate change based on the known structure of IGFBP-5 (see, e.g., Hwa et al., Endocrine Rev. (1999) 20:761-787.

By “fragment” is intended a polypeptide consisting of only a part of the intact, full-length polypeptide sequence and structure which retains biological activity as described above. The fragment can include a C-terminal deletion and/or an N-terminal deletion of the native polypeptide. A “fragment” of an IGFBP-5 will generally include at least about 10-250, such as 25-150 contiguous amino acid residues of the full-length molecule, preferably at least about 50-250 contiguous amino acid residues of the full-length molecule, and most preferably at least about 100-150 or more contiguous amino acid residues of the full-length molecule, or any integer between 10 amino acids and the full-length sequence, provided that the fragment in question retains biological activity as described herein. A heparin-binding domain is believed to occur at about positions 130-143 of the IGFBP-5 molecule depicted in FIGS. 1A-1B. This domain may be important for osteogenesis. Thus, fragments which include this span of amino acids are particularly preferred. Additionally, IGFBP-5 polypeptides are contemplated that include a C-terminal truncation occurring at a point between amino acid 100 and the C-terminus, preferably amino acid 143 and the C-terminus (numbered relative to the mature, full-length sequence depicted in FIGS. 1A-1B), such as a truncation occurring after amino acid 100 . . . 110 . . . 112 . . . 120 . . . 125 . . . 135 . . . 143,144,145 . . . 150 . . . 165 . . . 169 . . . 175 . . . 180 . . . 185 . . . 188, as well as any integers that fall between these stated numbers. For a description of certain fragments of IGFBP-5, see, e.g., Standker et al., FEBS Lett. (1998) 441:281-286. A particularly preferred C-terminal truncation occurs after amino acid 169, numbered relative to the full-length sequence depicted in FIGS. 1A-1B, to render IGFBP-5₁₋₁₆₉. This C-terminally truncated IGFBP-5 is described in, e.g., International Publication No. WO 94/10207, published May 11, 1994 (numbered 21-189 therein). Another active IGFBP-5 fragment includes a molecule with both a C-terminal and N-terminal truncation, IGFBP-5₄₋₁₄₃, also described in International Publication No. WO 94/10207 (numbered 24-163 therein). Other IGFBP-5 fragments with both N- and C-terminal truncations are described in, e.g., Standker et al., FEBS Lett. (1998) 441:281-286. These examples of fragments, of course, are merely representative and are not meant to be limiting.

By “purified” and “isolated” is meant, when referring to a polypeptide or polynucleotide, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated polynucleotide which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

By a “recombinant IGFBP-5 polypeptide” is intended an IGFBP-5 polypeptide having biological activity, as measured using the techniques described above and which has been prepared by recombinant DNA techniques as described herein. In general, the gene coding for IGFBP-5 is cloned and then expressed in transformed organisms, as described further below. The host organism expresses the foreign gene to produce IGFBP-5 under expression conditions. It is particularly advantageous to produce IGFBP-5 polypeptides recombinantly as recombinant production generally allows for higher yields from less starting material, and renders a far purer product. Thus, the polypeptides of the invention can be produced in the absence of other molecules normally present in cells. For example, human IGFBP compositions free of any trace human protein contaminants can be readily obtained because the only human protein produced by a recombinant non-human host cell is the recombinant human IGFBP-5. Potential viral agents from natural sources and viral components pathogenic to humans are also avoided.

The term “polynucleotide” or “nucleic acid molecule” as used herein refers to a polymeric form of nucleotides of any length, either ribonucleotides or deoxyribonucleotides. This term refers only to the primary structure of the molecule and thus includes double- and single-stranded DNA and RNA. It also includes known types of modifications, for example, labels which are known in the art, methylation, “caps”, substitution of one or more of the naturally occurring nucleotides with an analog, intemucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example proteins (including for e.g., nucleases, toxins, antibodies, signal peptides, poly-L-lysine, etc.), those with intercalators (e.g., acridine, psoralen, etc.), those containing chelates (e.g., metals, radioactive metals, boron, oxidative metals, etc.), those containing alkylators, those with modified linkages (e.g., alpha anomeric nucleic acids, etc.), as well as unmodified forms of the polynucleotide.

The terms “recombinant DNA molecule,” or “recombinant polynucleotide” are used herein to refer to a polynucleotide of genomic, cDNA, semisynthetic, or synthetic origin which, by virtue of its origin or manipulation: (1) is not associated with all or a portion of a polynucleotide with which it is associated in nature, (2) is linked to a polynucleotide other than that to which it is linked in nature, or (3) does not occur in nature. Thus, the term encompasses “synthetically derived” nucleic acid molecules.

A “coding sequence” is a nucleic acid molecule which is translated into a polypeptide, usually via mRNA, when placed under the control of appropriate regulatory sequences. The boundaries of the coding sequence may be determined by a translation start codon at the 5′-terminus and a translation stop codon at the 3′-terminus. A coding sequence can include, but is not limited to, cDNA, and recombinant nucleotide sequences.

“Control sequences” refer to nucleic acid sequences which are necessary to effect the expression of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequences. The term “control sequences” is intended to include, at a minimum, all components necessary for expression of a coding sequence, and may also include additional components, for example, leader sequences and fission partner sequences.

A control element, such as a promoter, “directs the transcription” of a coding sequence in a cell when RNA polymerase will bind the promoter and transcribe the coding sequence into mRNA, which is then translated into the polypeptide encoded by the coding sequence.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

As used herein, the term “expression cassette” refers to a molecule comprising at least one coding sequence operably linked to a control sequence which includes all nucleotide sequences required for the transcription of cloned copies of the coding sequence and the translation of the mRNAs in an appropriate host cell. Such expression cassettes can be used to express eukaryotic genes in a variety of hosts such as bacteria, blue-green algae, plant cells, yeast cells, insect cells and animal cells. Under the invention, expression cassettes can include, but are not limited to, cloning vectors, specifically designed plasmids, viruses or virus particles. The cassettes may further include an origin of replication for autonomous replication in host cells, selectable markers, various restriction sites, a potential for high copy number and strong promoters.

By “vector” is meant any genetic element, such as a plasmid, phage, transposon, cosmid, chromosome, virus etc., which is capable of replication when associated with the proper control elements and which can transfer gene sequences between cells. Thus, the term includes cloning and expression vehicles, as well as viral vectors.

A cell has been “transformed” by an exogenous polynucleotide when the polynucleotide has been introduced inside the cell membrane. The exogenous polynucleotide may or may not be integrated (covalently linked) into chromosomal DNA making up the genome of the cell. In procaryotes and yeasts, for example, the exogenous DNA may be maintained on an episomal element, such as a plasmid. With respect to eucaryotic cells, a stably transformed cell is one in which the exogenous DNA has become integrated into the chromosome so that it is inherited by daughter cells through chromosome replication. This stability is demonstrated by the ability of the eucaryotic cell to establish cell lines or clones comprised of a population of daughter cells containing the exogenous DNA.

A “host cell” is a cell which has been transformed, or is capable of transformation, by an exogenous nucleic acid molecule.

“Homology” refers to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100.

Preferably, naturally or non-naturally occurring protein variants have amino acid sequences which are at least 85%, 90% or 95% identical to the amino acid sequence shown at positions 1-252 of FIGS. 1A-1B, or to a shorter portion of the sequence shown in FIGS. 1A-1B. More preferably, the molecules are 98% or 99% identical. Percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman, Adv. Appl. Math. 2:482-489 (1981).

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. For example, an effective amount of an IGFBP-5 polypeptide for use with the present methods is an amount sufficient to slow, stop or reverse the bone mineral density reduction rate, and preferably an amount sufficient to increase the bone formation rate and/or enhance bone accretion, in a patient suffering from Type I osteoporosis and vertebral or femoral fractures. Such amounts are described below. An appropriate “effective” amount in any individual case may be determined by one of ordinary skill in the art using routine experimentation.

By “enhanced bone accretion” is meant that bone accumulation in a subject administered the IGFBP-5 polypeptides of the invention is increased over bone accumulation in a subject that is not given an IGFBP-5 polypeptide. Such enhanced bone accretion is determined herein by measuring BMD.

By “increased bone formation” is meant that the rate of bone formation in a subject administered the IGFBP-5 polypeptides of the invention is increased over the bone formation rate in a subject that is not given an IGFBP-5 polypeptide. Such enhanced bone formation is determined herein using, e.g., quantitative digitized morphometry, as well as by other markers of bone formation, as described above.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiological range” is meant a pH in the range of approximately 7.2 to 8.0 inclusive, more typically in the range of approximately 7.2 to 7.6 inclusive.

As used herein, the term “subject” encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of compositions and methods similar or equivalent to those described herein can be used in the practice of the present invention, the preferred materials and methods are described herein.

The present invention is based on the discovery that IGFBP-5 polypeptides, including native, mature, full-length IGFBP-5, as well as biologically active analogs and fragments thereof, are able to increase bone formation and enhance bone accretion in vivo and are therefore useful for treating osteoporosis caused by estrogen deficiencies. As explained above, Type I osteoporosis refers to a condition where bone loss is accelerated due to estrogen deficiency. This type of osteoporosis is seen in postmenopausal women, women who lack ovaries and in people with certain endocrine disorders that result in low circulating estrogen levels and osteoporosis. The main clinical manifestations of Type I osteoporosis are vertebral, femoral and Colles' fractures. Bone resorption is increased in Type I osteoporosis but there is inadequate bone formation. Accordingly, the present invention provides a means to compensate for inadequate bone formation by directly stimulating osteoblast activity and bone formation.

The method uses compositions comprising IGFBP-5 polypeptides, as discussed above, with or without added bone formation enhancers or bone resorption inhibitors. Such substances include, without limitation, IGF-1, IGF-2, estrogen, calcitonin, bisphosphonates, parathyroid hormone (PTH), and estrogen receptor modulators.

IGFBP-5 polypeptides for use with the subject methods can be isolated directly from a tissue or organ that produces the same, using techniques well known in the art. Methods for purifying native, full-length IGFBP-5 from serum are described in, for example, International Publication No. WO 92/14834. Procedures for purifying a carboxyl-truncated variant of IGFBP-5, from osteosarcoma cells are described in, e.g., International Publication No. WO 94/10207 and Andress and Birnbaum, J. Biol. Chem. (1992) 267:22467-22472.

Alternatively, polypeptides for use in the subject methods can be synthesized chemically, by any of several techniques that are known to those skilled in the peptide art. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. The polypeptides of the present invention can also be chemically prepared by the method of simultaneous multiple peptide synthesis. See, e.g., Houghten Proc. Natl. Acad. Sci. USA (1985) 82:5131-5135; U.S. Pat. No. 4,631,211.

Preferably, polypeptides for use in the present methods are produced recombinantly, by expression of a polynucleotide encoding the same. Methods for the recombinant production of IGFBP-5 polypeptides are well known. See, e.g., International Publication No. WO 92/14834; Kiefer et al.,, Biochem. Biophys. Res. Commun. (1991) 176:219-225; and Allander et al., J. Biol. Chem. (1994) 269:10891-10898 for methods of recombinantly producing full-length IGFBP-5 polypeptides; and Andress et al., Biochem. Biophys. Res. Commun. (1993) 195:25-30, for methods of recombinantly producing a truncated IGFBP-5 polypeptide (IGFBP-5¹⁻¹⁶⁹).

In particular, these and other molecules for use with the present invention can be made using standard techniques of molecular biology. For example, polynucleotide sequences coding for the above-described molecules can be obtained using recombinant methods, such as by screening cDNA and genomic libraries from cells expressing the gene, or by deriving the gene from a vector known to include the same. Furthermore, the desired gene can be isolated directly from cells and tissues containing the same, using standard techniques, such as phenol extraction and PCR of cDNA or genomic DNA. See, e.g., Sambrook et al., supra, for a description of techniques used to obtain and isolate DNA. The gene of interest can also be produced synthetically, rather than cloned. The molecules can be designed with appropriate codons for the particular sequence. The complete sequence is then assembled from overlapping oligonucleotides prepared by standard methods and assembled into a complete coding sequence. See, e.g., Edge (1981) Nature 292:756; Nambair et al. (1984) Science 223:1299; and Jay et al. (1984) J. Biol. Chem. 259:6311.

Thus, particular nucleotide sequences can be obtained from vectors harboring the desired sequences or synthesized completely or in part using various oligonucleotide synthesis techniques known in the art, such as site-directed mutagenesis and polymerase chain reaction (PCR) techniques where appropriate. See, e.g., Sambrook, supra. In particular, one method of obtaining nucleotide sequences encoding the desired sequences is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al. (1991) Proc. Natl. Acad. Sci. USA 88:4084-4088. Additionally, oligonucleotide directed synthesis (Jones et al. (1986) Nature 54:75-82), oligonucleotide directed mutagenesis of pre-existing nucleotide regions (Riechmann et al. (1988) Nature 332:323-327 and Verhoeyen et al. (1988) Science 239:1534-1536), and enzymatic filling-in of gapped oligonucleotides using T₄ DNA polymerase (Queen et al. (1989) Proc. Natl. Acad. Sci. USA 86:10029-10033) can be used under the invention to provide molecules having altered or enhanced antigen-binding capabilities, and/or reduced immunogenicity.

Once coding sequences have been prepared or isolated, such sequences can be cloned into any suitable vector or replicon. Numerous cloning vectors are known to those of skill in the art, and the selection of an appropriate cloning vector is a matter of choice. Suitable vectors include, but are not limited to, plasmids, phages, transposons, cosmids, chromosomes or viruses which are capable of replication when associated with the proper control elements.

The coding sequence is then placed under the control of suitable control elements, depending on the system to be used for expression. Thus, the coding sequence can be placed under the control of a promoter, ribosome binding site (for bacterial expression) and, optionally, an operator, so that the DNA sequence of interest is transcribed into RNA by a suitable transformant. The coding sequence may or may not contain a sequence coding for a signal peptide or leader sequence which can later be removed by the host in post-translational processing. See, e.g., U.S. Pat. Nos. 4,431,739; 4,425,437; 4,338,397. If a signal sequence is present, it can either be the native sequence shown at positions −20 through −1 of FIGS. 1A-1B, or it may be a heterologous signal sequence.

In addition to control sequences, it may be desirable to add regulatory sequences which allow for regulation of the expression of the sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector. For example, enhancer elements may be used herein to increase expression levels of the constructs. Examples include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777) and elements derived from human CMV (Boshart et al. (1985) Cell 41:521), such as elements included in the CMV intron A sequence (U.S. Pat. No. 5,688,688). The expression cassette may further include an origin of replication for autonomous replication in a suitable host cell, one or more selectable markers, one or more restriction sites, a potential for high copy number and a strong promoter.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the molecule of interest may be desirable to achieve this end. For example, in some cases it may be necessary to modify the sequence so that it can be attached to the control sequences in the appropriate orientation; i.e., to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site.

As explained above, it may also be desirable to produce mutants or analogs of the IGFBP-5 polypeptide. Mutants or analogs may be prepared by the deletion of a portion of the sequence encoding the IGFBP-5 polypeptide, by insertion of a sequence, and/or by substitution of one or more nucleotides within the sequence. Techniques for modifying nucleotide sequences, such as site-directed mutagenesis, and the like, are well known to those skilled in the art. See, e.g., Sambrook et al., supra; Kunkel, T. A. (1985) Proc. Natl. Acad. Sci. USA (1985) 82:448; Geisselsoder et al. (1987) BioTechniques 5:786; Zoller and Smith (1983) Methods Enzymol. 100:468; Dalbie-McFarland et al. (1982) Proc. Natl. Acad. Sci USA 79:6409.

The molecules can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells, human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodopterafrugiperda, and Trichoplusia ni.

Nucleic acid molecules comprising nucleotide sequences of interest can be stably integrated into a host cell genome or maintained on a stable episomal element in a suitable host cell using various gene delivery techniques well known in the art. See, e.g., U.S. Pat. No. 5,399,346.

Depending on the expression system and host selected, the molecules are produced by growing host cells transformed by an expression vector described above under conditions whereby the protein is expressed. The expressed protein is then isolated from the host cells and purified. If the expression system secretes the protein into growth media, the product can be purified directly from the media. If it is not secreted, it can be isolated from cell lysates. The selection of the appropriate growth conditions and recovery methods are within the skill of the art.

The IGFBP-5 polypeptide (with or without additional bone enhancers or resorption retardants) may be formulated into pharmaceutical compositions, described further below, for delivery to a subject. Alternatively, polynucleotides encoding the polypeptide for use in the methods of the present invention may be delivered directly to the subject and expressed in vivo. When a polynucleotide encoding the IGFBP-5 polypeptide is delivered, it may be desirable to administer a polynucleotide encoding a fusion protein, such as a fusion between a signal sequence (homologous or heterologous) and the IGFBP-5 polypeptide.

A number of viral based systems have been developed for direct gene transfer into mammalian cells. In this regard, retroviruses provide a convenient platform for gene delivery systems. A selected nucleotide sequence encoding the desired polypeptide can be inserted into a vector and packaged in retroviral particles using techniques known in the art. The recombinant virus can then be isolated and delivered to a subject. A number of suitable retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

A number of suitable adenovirus vectors have also been described. Unlike retroviruses which integrate into the host genome, adenoviruses persist extrachromosomally thus minimizing the risks associated with insertional mutagenesis (Haj-Ahmad and Graham (1986) J, Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) Biotechniques 6:616-629; and Rich et al. (1993) Human Gene Therapy 4:461-476). Various adeno-associated virus (AAV) vector systems have been developed recently for gene delivery. Such systems can include control sequences, such as promoter and polyadenylation sites, as well as selectable markers or reporter genes, enhancer sequences, and other control elements which allow for the induction of transcription. AAV vectors can be readily constructed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors which will find use for delivering the nucleic acid molecules encoding the molecules of the present invention by gene transfer include those derived from the pox family of viruses, such as vaccinia virus and avian poxvirus. See, e.g., International Publication Nos. WO 91/12882; WO 89/03429; and WO 92/03545.

Molecular conjugate vectors, such as the adenovirus chimeric vectors described in Michael et al. (1993) J. Biol. Chem. 268:6866-6869 and Wagner et al. (1992) Proc. Natl. Acad. Sci. USA 89:6099-6103, can also be used for gene delivery under the invention.

Members of the Alphavirus genus, such as but not limited to vectors derived from the Sindbis and Semliki Forest viruses, will also find use as viral vectors for delivering the gene of interest. For a description of Sinbus-virus derived vectors useful for the practice of the instant methods, see, Dubensky et al., J. Virol. (1996) 70:508-519; and International Publication Nos. WO 95/07995 and WO 96/17072.

The gene of interest can also be delivered without a viral vector. For example, the gene can be packaged in liposomes prior to delivery to the subject or to cells derived therefrom, with or without the accompanying antigen. Lipid encapsulation is generally accomplished using liposomes which are able to stably bind or entrap and retain nucleic acid. For a review of the use of liposomes as carriers for delivery of nucleic acids, see, Hug and Sleight, Biochim. Biophys. Acta. (1991) 1097:1-17; Straubinger et al., in Methods of Enzymology (1983), Vol. 101, pp. 512-527.

The method uses pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, etc. Suitable excipients for nonliquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Once formulated, the compositions of the invention are generally administered parenterally. Administration can include, for example, administration intravenously, intra-arterially, intra-articularly (e.g., into the knee), subcutaneously, intradermally, intramuscularly, transdermally, intranasally, mucosally, and by aerosol administration. For example, the composition can be administered by inhalation, e.g., as a nasal or mouth spray or aerosol. The compositions may also be delivered in situ, e.g., by implantation.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the present invention, generally a therapeutic amount will be in the range of about 0.1 mg/kg to about 40 mg/kg body weight, more preferably about 0.5 mg/kg to about 20 mg/kg, in at least one dose. In larger mammals, for example humans, the indicated daily dosage can be from about 5 mg to 100 mg, one or more times per day. Typically, if polynucleotides are delivered, the doses will be at least an order of magnitude lower. The subject may be administered as many doses as is required to reduce and/or alleviate the signs, symptoms, or causes of the disorder in question, or bring about any other desired alteration of a biological system.

III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE

### Ability of IGFBP-5 Polypeptides to Increase Bone Formation and Accretion in vivo

In order to determine the ability of IGFBP-5 polypeptides to stimulate bone formation and bone accretion in vivo, polypeptides were tested in an established mouse model of estrogen deficiency. In particular, three month old, C3H (inbred) mice were ovariectomized (OVX) or sham operated, one month before beginning IGFBP-5 injections. Recombinant human, full-length IGFBP-5 (“IGFBP-5^{intact}”) was produced as described in International Publication No. WO 92/14834. A C-terminally truncated molecule, IGFBP-5¹⁻¹⁶⁹, was produced recombinantly as described in Andress et al., Biochem. Biophys. Res. Commun. (1993) 195:25-30. These IGFBP-5 polypeptides were dissolved in phosphate buffered saline (PBS) and frozen in aliquots. All four groups of mice (n=10-12/group) received daily subcutaneous injections of either IGFBP-5^{intact}, IGFBP-5¹⁻¹⁶⁹, or PBS for 8 weeks. Bone mineral density (BMD) of the spine and femur was determined at baseline and every two weeks by dual energy x-ray absorptiometry. After 7 weeks of treatment, the mice received tetracycline injections for bone labeling 7 days and again 3 days before sacrifice. Bone from the lumbar spine and femur were harvested for histologic studies and bone histology was performed on un-decalcified, methyl-methacrylate embedded sections. Dynamic measurements of bone formation rate (BFR) were performed on tetracycline labeled cancellous bone from the lumbar spine and distal femur metaphysis using quantitative digitized morphometry.

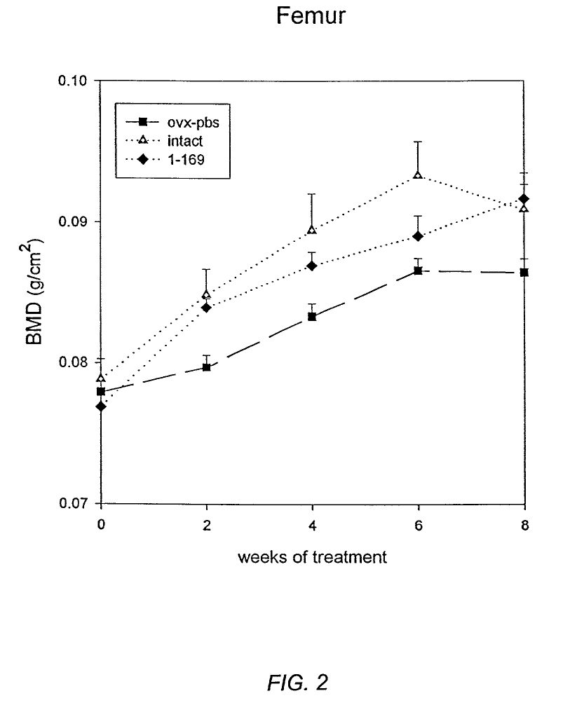

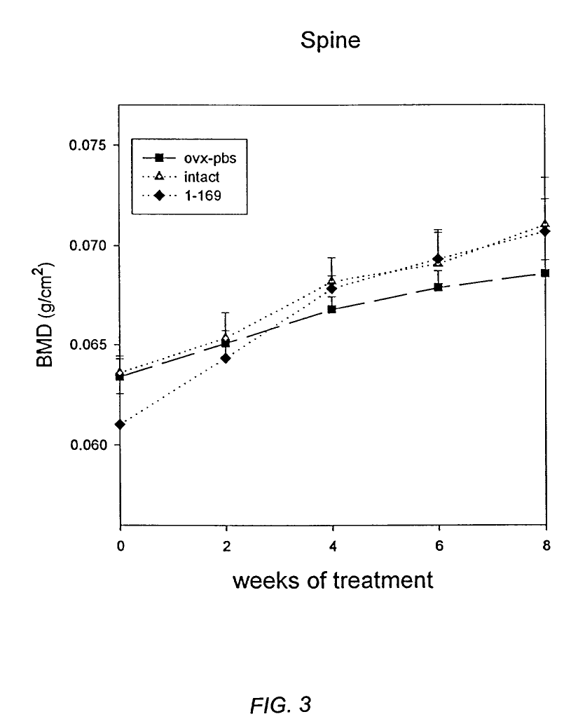

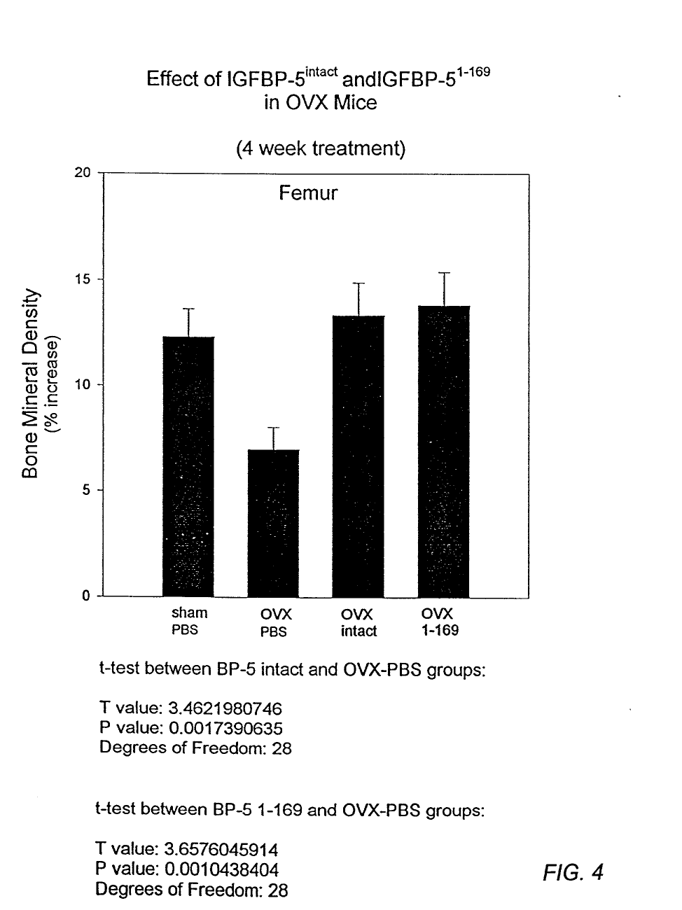

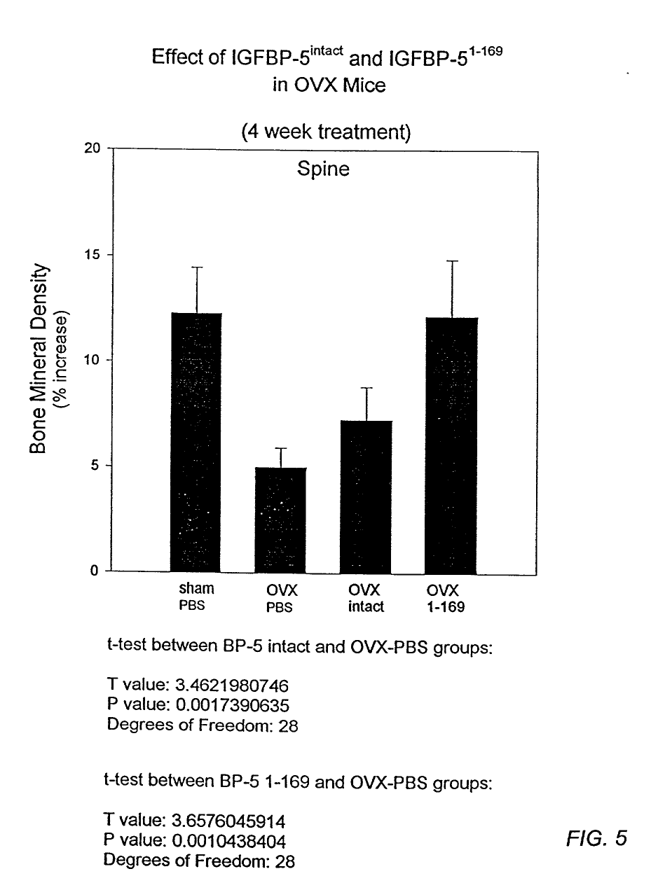

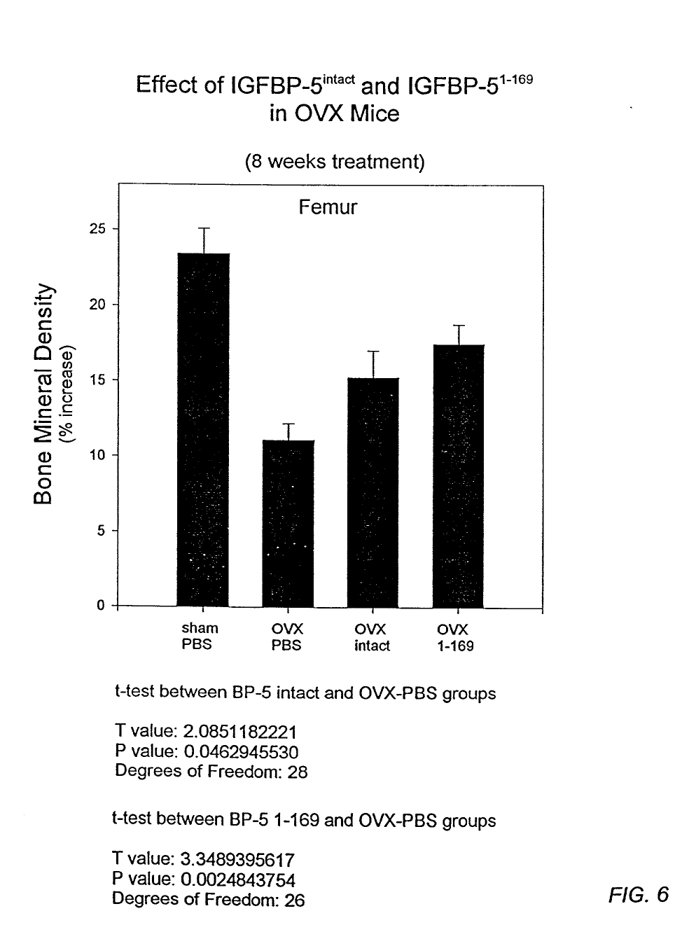

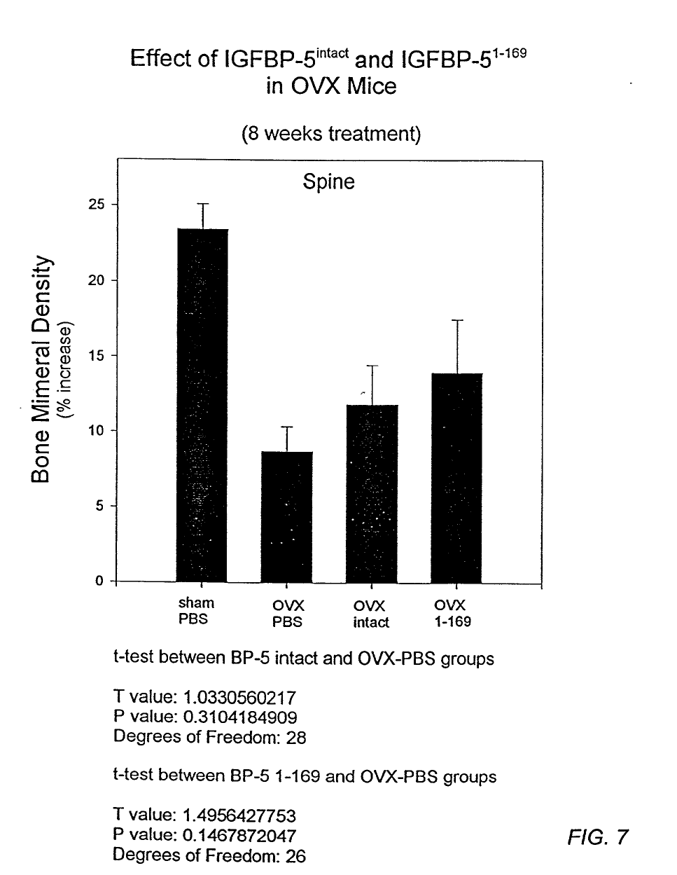

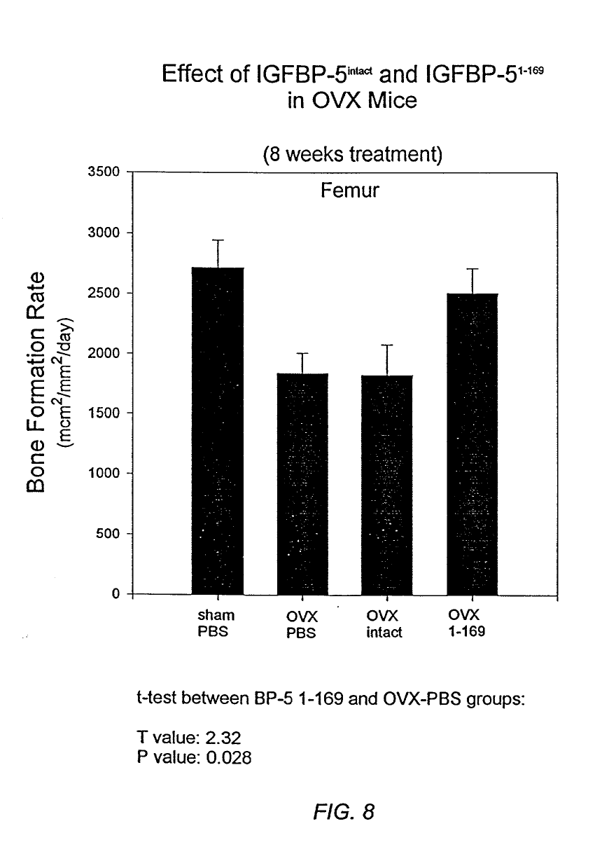

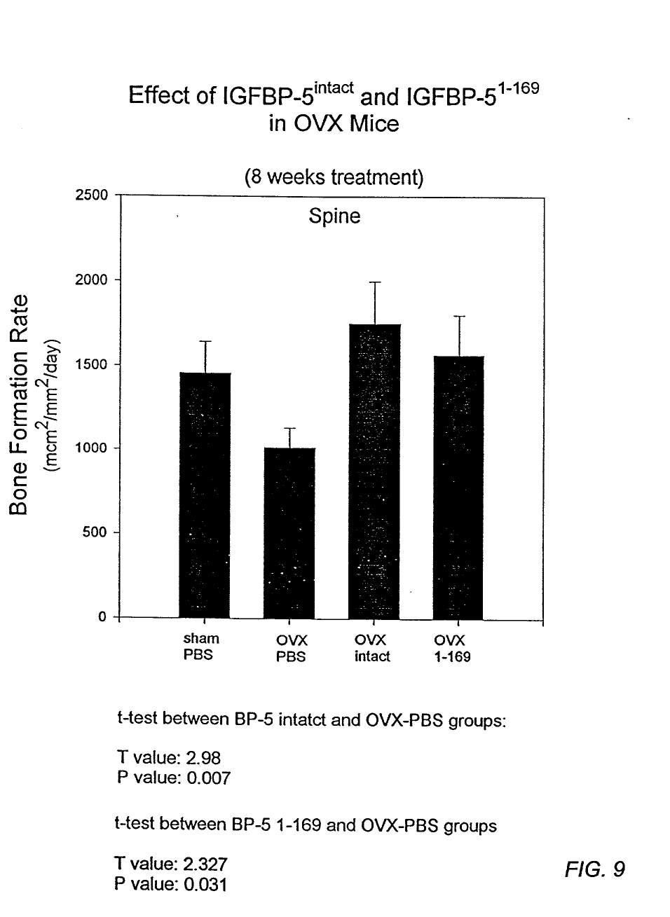

As can be seen in FIGS. 2-5, After four weeks of treatment, the OVX-control group had gained 45% less BMD in the femur and 50% less in the spine than the sham-controls, indicating that estrogen deficiency decreases bone accretion in growing mice. In contrast, OVX mice treated with IGFBP-5^{intact} and IGFBP-5¹⁻¹⁶⁹ had identical gains in femur BMD compared to sham controls. The spine BMD accretion rate was also completely preserved in the IGFBP-5¹⁻¹⁶⁹ group but not in the IGFBP-5^{intact} treated mice. After 8 weeks of treatment (FIGS. 6 and 7) femur bone accretion rates were 36% greater in the IGFBP-5^{intact} group (p<0.05) and 54% greater in the IGFBP-5¹⁻¹⁶⁹ group (p<0.002) compared to OVX-controls though they were significantly less than the sham-controls. Spine and femur BFR, after 8 weeks of treatment, were reduced by 30% in the OVX-controls (p<0.03). Femur BFR in the IGFBP-5^{intact} group was identical to OVX-controls whereas in the IGFBP-5¹⁻¹⁶⁹ treated group it was greater than the OVX-controls (p<0.03) and not significantly different from the sham controls. Spine BFR in both IGFBP-5 groups were identical to sham controls and were significantly greater than OVX-controls (P<0.03). (See, FIGS. 8 and 9.) There were no significant differences in weight among the 4 treatment groups.

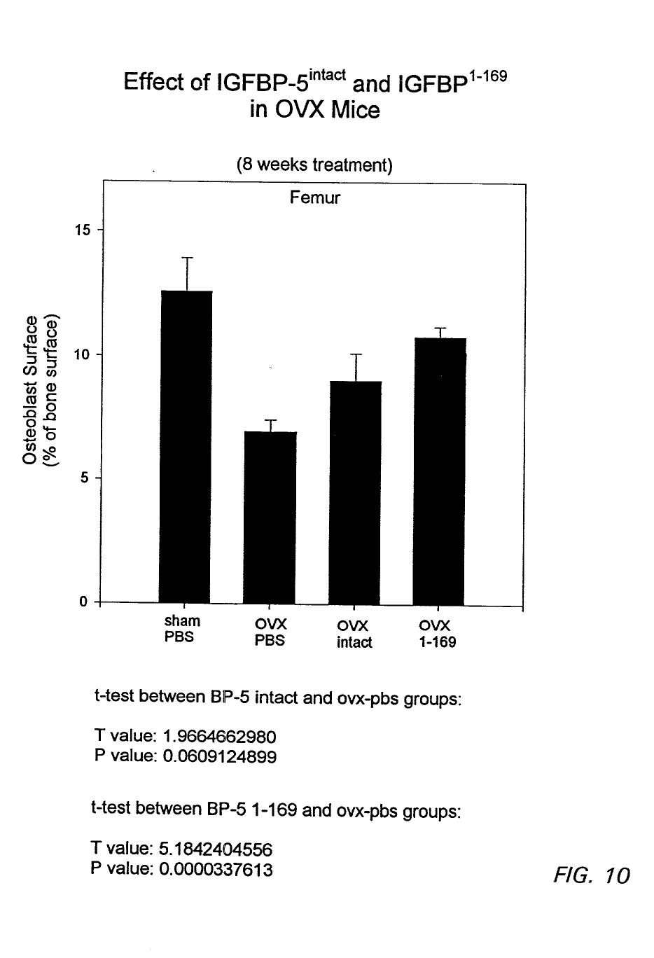

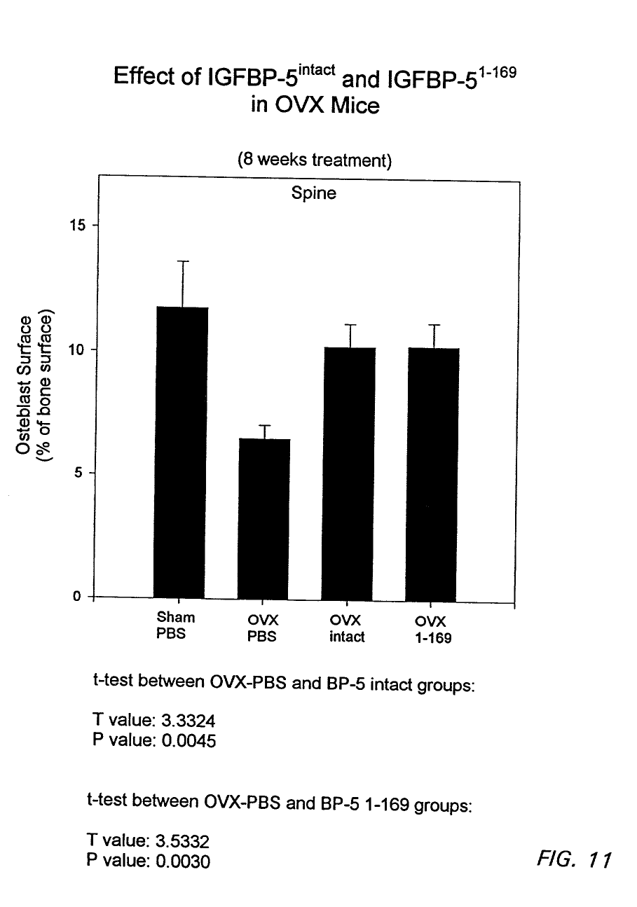

As can be seen in FIGS. 10 and 11, spine and femur osteoblast surfaces, after 8 weeks of treatment, were reduced by 45% in the OVX controls (p<0.005) indicating that estrogen deficiency reduces osteoblast number in growing mice. Femur osteoblast surface in the IGFBP-5^{intact} group was not statistically different from the OVX-controls (p=0.06 1) whereas in the IGFBP-5¹⁻¹⁶⁹ treated group it was significantly greater than the OVX control (p<0.003) and not different from sham controls. Spine osteoblast surface in both the IGFBP-5^{intact} and IGFBP-5¹⁻¹⁶⁹ treated groups was greater than the OVX controls (p<0.005) and not significantly different from sham controls.

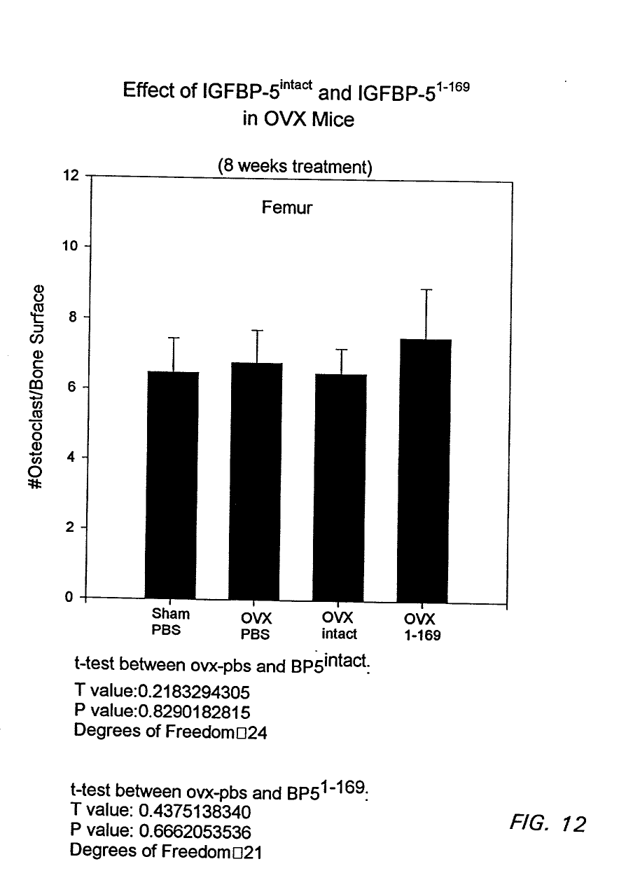

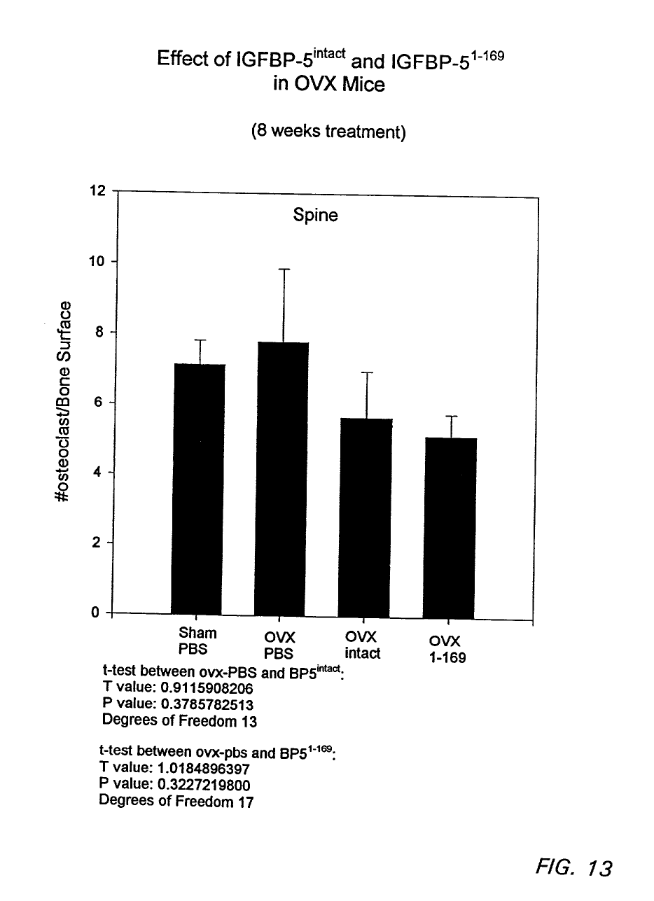

As can be seen in FIGS. 12 and 13, osteoclast surfaces were not altered by ovariectomy-induced estrogen deficiency and treatments with IGFBP-5^{intact} or IGFBP-5¹⁻¹⁶⁹ did not affect osteoclast number.

As seen above, estrogen deficiency in mice, induced by OVX, resulted in lower than normal bone accretion which was due primarily to decreased osteoblast number and decreased bone formation. IGFBP-5¹⁻¹⁶⁹ stimulated osteoblast number and bone formation in the femur and spine whereas IGFBP-5^{intact} increased only spine osteoblast number and bone formation in OVX mice after 8 weeks of treatment. Bone accretion in the femur was completely preserved by IGFBP-5¹⁻¹⁶⁹ and IGFBP-5^{intact} after 4 weeks and by 50% and 33% by IGFBP-5¹⁻¹⁶⁹ and IGFBP-5^{intact}, respectively, after 8 weeks of treatment. Bone accretion in the spine was completely preserved by IGFBP-5¹⁻¹⁶⁹ and 39% preserved by IGFBP-5^{intact} after 4 weeks. After 8 weeks of treatment spine accretion rates were slightly greater in both IGFBP-5 treatment groups but were not statistically significant.

Thus, IGFBP-5 treatment prevents reductions in osteoblast number and bone formation and enhances bone accretion in estrogen deficient mice. IGFBP-5 treatment affects both the spine and femur but appears to be more beneficial in the femur than the spine. Both the full-length and truncated molecules display these effects, however, IGFBP-5¹⁻¹⁶⁹ may be more effective than IGFBP-5^{intact}, particularly in the femur. IGFBP-5 treatment does not affect osteoclast number in estrogen-deficient mice.

Thus, novel methods for enhancing bone formation and accretion are disclosed. Although preferred embodiments of the subject invention have been described in some detail, it is understood that obvious variations can be made without departing from the spirit and the scope of the invention as defined by the appended claims.

We claim:

1. A method for increasing bone formation and/or enhancing bone accretion in a subject who has Type I osteoporosis, said method comprising administering to the subject a pharmaceutically effective amount of an IGFBP-5 polypeptide.

2. The method of claim 1 wherein the IGFBP-5 polypeptide is a full-length IGFBP-5 molecule.

3. The method of claim 2, wherein the IGFBP-5 molecule comprises the sequence of amino acids depicted at positions 1-252, inclusive of FIGS. 1A-1B, or a molecule with 75% identity thereto.

4. The method of claim 3, wherein the IGFBP-5 molecule comprises the sequence of amino acids depicted at positions 1-252, inclusive of FIGS. 1A-1B.

5. The method of claim 1, wherein the IGFBP-5 is a C-terminally truncated fragment of the IGFBP-5 depicted at amino acid positions 1-252, inclusive, of FIGS. 1A-1B, or a C-terminally truncated fragment of a molecule with 75% identity to the IGFBP-5 depicted at amino acid positions 1-252, inclusive, in FIGS. 1A-1B, wherein said C-terminal truncation occurs at an amino acid after amino acid 100.

6. The method of claim 1, wherein the IGFBP-5 is a C-terminally truncated fragment of the IGFBP-5 depicted at amino acid positions 1-252, inclusive, of FIGS. 1A-1B, or a C-terminally truncated fragment of a molecule with 75% identity to the IGFBP-5 depicted at amino acid positions 1-252, inclusive, in FIGS. 1A-1B, wherein said C-terminal truncation occurs at an amino acid after amino acid 143.

7. The method of claim 6, wherein the C-terminally truncated fragment of the IGFBP-5 comprises amino acids 1-169, inclusive, of FIGS. 1A-1B, or a sequence of amino acids with 75% identity to the contiguous sequence of amino acids 1-169 of FIGS. 1A-1B.

8. The method of claim 6, wherein the C-terminally truncated fragment of the IGFBP-5 consists of amino acids 1-169, inclusive, of FIGS. 1A-1B.

9. The method of claim 1, further comprising administering an additional bone formation enhancer to the subject.

10. The method of claim 1, further comprising administering a bone resorption inhibitor to the subject.

11. The method of claim 1, wherein said IGFBP-5 polypeptide increases bone formation in said subject.

12. The method of claim 1, wherein said IGFBP-5 polypeptide enhances bone accretion in said subject.

13. A method for increasing bone formation and/or enhancing bone accretion in a subject who has estrogen deficiency-related Type I osteoporosis, said method comprising administering to the subject a pharmaceutically effective amount of an IGFBP-5 polypeptide comprising amino acids 1-252, inclusive, of FIGS. 1A-1B.

14. A method for increasing bone formation and/or enhancing bone accretion in a subject who has estrogen deficiency-related Type I osteoporosis, said method comprising administering to the subject a pharmaceutically effective amount of a C-terminally truncated IGFBP-5 fragment comprising amino acids 1-169, inclusive, of FIGS. 1A-1B.

15. The method of claim 14, wherein the C-terminally truncated IGFBP-5 fragment consists of amino acids 1-169, inclusive, of FIGS. 1A-1B.

16. A method for increasing bone formation and/or enhancing bone accretion in a subject who has estrogen deficiency-related Type I osteoporosis, said method comprising administering to the subject a pharmaceutically effective amount of a polynucleotide encoding an IGFBP-5 polypeptide.


SEQUENCE LISTING (2 sequences)

SEQ ID NO: 1 - protein, 272 aa, Artificial Sequence
  Description of Artificial Sequence  human IGFBP-5
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHTFDSSNVE

SEQ ID NO: 2 - DNA, 816 bp, Artificial Sequence
  Description of Artificial Sequence  human IGFBP-5
atggtgttgctcaccgcggtcctcctgctgctggccgcctatgcggggccggcccagagcctgggctccttcgtgcactgcgagccctgcgacgagaaagccctctccatgtgcccccccagccccctgggctgcgagctggtcaaggagccgggctgcggctgctgcatgacctgcgccctggccgaggggcagtcgtgcggcgtctacaccgagcgctgcgcccaggggctgcgctgcctcccccggcaggacgaggagaagccgctgcacgccctgctgcacggccgcggggtttgcctcaacgaaaagagctaccgcgagcaagtcaagatcgagagagactcccgtgagcacgaggagcccaccacctctgagatggccgaggagacctactcccccaagatcttccggcccaaacacacccgcatctccgagctgaaggctgaagcagtgaagaaggaccgcagaaagaagctgacccagtccaagtttgtcgggggagccgagaacactgcccacccccggatcatctctgcacctgagatgagacaggagtctgagcagggcccctgccgcagacacatggaggcttccctgcaggagctcaaagccagcccacgcatggtgccccgtgctgtgtacctgcccaattgtgaccgcaaaggattctacaagagaaagcagtgcaaaccttcccgtggccgcaagcgtggcatctgctggtgcgtggacaagtacgggatgaagctgccaggcatggagtacgttgacggggactttcagtgccacaccttcgacagcagcaacgttgag